(19) United States
(12) Patent Application Publication
(10) Pub. No.: US 2010/0312148 A1
(43) Pub. Date: Dec. 9, 2010

(54) POSTURE EVALUATING APPARATUS

(75) Inventor: Tetsuya Sato, Kyoto-shi (JP)

Correspondence Address: OLIFF & BERRIDGE, PLC, P.O. BOX 320850, ALEXANDRIA, VA, 22320-4850, US

(73) Assignee: Omron Healthcare Co., Ltd., Kyoto-shi, Kyoto (JP)

(21) Appl. No.: 12/866,792

(22) PCT Filed: Mar. 9, 2009
(86) PCT No.: PCT/JP2009/054424

§ 371 Date: Aug. 9, 2010

(30) Foreign Application Priority Data
Mar. 14, 2008 (JP) 2008-066463

(51) Int. Cl. A61B 5/103

(52) U.S. Cl. 600/594

(57) ABSTRACT

A posture evaluating apparatus according to the invention includes: a grasp portion that is grasped with user's hands; a pedestal portion on which the user rides with both feet; inclination detecting means for detecting inclination of the grasp portion; barycentric position detecting means for detecting a barycentric position of a load acting on the pedestal portion; posture evaluating means for evaluating posture of the user based on the inclination of the grasp portion and the barycentric position of the load acting on the pedestal portion; and output means for outputting evaluation result of the posture evaluating means.

### TECHNICAL FIELD

The present invention relates to an apparatus that evaluates a posture of a user.

### BACKGROUND ART

Conventionally, particular emphasis is placed on maintaining good posture from the viewpoints of life and health. For example, it is said that not only the bad posture looks strange, but also the bad posture leads to strain of a body, weakening eyesight, weakening internal organ function, shoulder stiffness, lower back ache, headache, fatigue, and the like. On the other hand, it is said that not only the good posture looks beautiful, but also the good posture improves dental bite, sleeping, and the like. Therefore, there is a demand for a technique of evaluating the posture and the strain of the skeleton.

For example, Patent Documents 1 to 3 disclose the technique related to the posture evaluation. Patent Document 1 discloses a body fat scale that starts the measurement when a hand electrode portion (grip portion of body fat scale) is kept in a horizontal position. Patent Document 2 discloses a weight scale that has a function of measuring a posture balance (which direction a barycenter of the body is biased toward) with a load sensor. Patent Document 3 discloses a posture diagnostic facility including an imaging apparatus that images a posture of a subject and a foot pressure measuring apparatus that measures a foot pressure of the subject.

However, the body fat scale disclosed in Patent Document 1 is a technique of retaining both arms at a desired angle during the measurement. Although the weight scale disclosed in Patent Document 2 can evaluate posture balance, the weight scale cannot evaluate the strain of the body. The posture diagnostic facility disclosed in Patent Document 3 becomes a massive one because the imaging apparatus that images the posture of the subject is required. Therefore, the posture cannot simply be diagnosed.

Patent Document 1: Japanese Unexamined Patent Publication No. 2001-149328

Patent Document 2: Japanese Unexamined Patent Publication No. 2003-339671

Patent Document 3: Japanese Unexamined Patent Publication No. 2005-224452

### DISCLOSURE OF THE INVENTION

### Problems to be Solved by the Invention

In the apparatus (for example, weight scale disclosed in Patent Document 2) that evaluates the posture using only a barycentric position of a user, it is evaluated that the posture is good when the barycentric position of the user is located in the center (barycentric position in the good posture). However, occasionally the barycentric position becomes the center depending on the way the user is standing even if the user's body strains (for example, right shoulder slopes downward). The apparatus that evaluates the posture using only the barycentric position of the user makes a false evaluation that the user posture is good.

In view of the foregoing, an object of the invention is to provide a posture evaluating apparatus that can effectively assist the improvement of the user posture while evaluating the user posture by the simple method.

### Means for Solving the Problem

The invention adopts the following configurations in order to achieve the object.

A posture evaluating apparatus according to the invention is characterized by including: a grasp portion that is grasped with user's hands; a pedestal portion on which the user rides with both feet; inclination detecting means for detecting inclination of the grasp portion; barycentric position detecting means for detecting a barycentric position of a load acting on the pedestal portion; posture evaluating means for evaluating posture of the user based on the inclination of the grasp portion and the barycentric position of the load acting on the pedestal portion; and output means for outputting evaluation result of the posture evaluating means.

According to the configuration, the posture can be evaluated by the extremely simple method in which the user posture is evaluated based on the inclination of the grasp portion and the barycentric position (barycentric position of the user) of the load acting on the pedestal portion. Additionally, according to the configuration, the user posture is evaluated based on the inclination of the grasp portion and the barycentric position of the load acting on the pedestal portion, so that the detailed evaluation result can be obtained compared with the posture evaluation that is obtained only by the inclination of the grasp portion or the barycentric position of the load acting on the pedestal portion. For example, because the inclination of the grasp portion can be considered as a deviation of user's shoulder (for example, right shoulder slopes downward), the evaluation of the bad posture can be made when the inclination of the grasp portion is deviated even if the barycentric position is located in the center. That is, the false evaluation generated by the conventional method for evaluating the posture using only the barycentric position of the load acting on the pedestal portion can be reduced. Therefore, the improvement of the user posture can effectively be assisted.

Preferably the barycentric position detecting means is a load sensor that is provided in the pedestal portion. Preferably the inclination detecting means is an inclination sensor that is provided in the grasp portion. According to the configuration, the posture evaluating apparatus can easily be configured.

Preferably the posture evaluating means evaluates strain of a body based on the inclination of the grasp portion and the barycentric position of the load acting on the pedestal portion. According to the configuration, the user can recognize the own posture in detail. Therefore, the improvement of the user posture can effectively be assisted.

Preferably the output means outputs advice to have good posture according to the evaluation result of the posture evaluating means. According to the configuration, the user can immediately recognize what the user should do in order to have the good posture. Therefore, the improvement of the user posture can effectively be assisted.

Preferably the posture evaluating apparatus further includes body composition estimating means for measuring impedance of the user to estimate a body composition from the impedance. According to the configuration, the user can also obtain pieces of information on the weight and the body composition such as a body fat percentage. Actually it is said that the body shape and the posture are related to each other, and it is believed that the body shape is improved by having the good posture. Therefore, the user can recognize the effect of having the good posture by obtaining the pieces of information on the weight and the body composition. Accordingly, motivation in the improvement of the user posture can be expected to be enhanced. The user can be caused to have consciousness of not only the improvement of the posture but also the improvement of the body shape by obtaining the pieces of information on the weight and the body composition.

### EFFECT OF THE INVENTION

According to the invention, the user posture can be evaluated in detail by the simple method, and the improvement of the user posture can effectively be assisted.

### BRIEF DESCRIPTION OF THE DRAWINGS

FIG. 1 is a schematic diagram of a posture evaluating apparatus according to first to third embodiments.

FIG. 2 is a block diagram illustrating an internal configuration of the posture evaluating apparatus according to the first to third embodiments.

FIG. 3 is a flowchart illustrating a measurement flow of the posture evaluating apparatus according to the first to third embodiments.

FIG. 4 is a view illustrating an example of a posture evaluating method performed by the posture evaluating apparatus according to the first embodiment.

FIG. 5 is a view illustrating an example of an evaluation result displaying method performed by the posture evaluating apparatus according to the first embodiment.

FIG. 6 is a view illustrating an example of a posture evaluating method performed by the posture evaluating apparatus according to the second embodiment.

FIG. 7 is a view illustrating an example of an evaluation result displaying method performed by the posture evaluating apparatus according to the second embodiment.

FIG. 8 is a view illustrating an example of an advice displaying method performed by the posture evaluating apparatus according to the third embodiment.

FIG. 9 is a schematic diagram of a posture evaluating apparatus according to a fourth embodiment.

FIG. 10 is a block diagram illustrating an internal configuration of the posture evaluating apparatus according to the fourth embodiment.

FIG. 11 is a flowchart illustrating a measurement flow of the posture evaluating apparatus according to the fourth embodiment.

### BEST MODES FOR CARRYING OUT THE INVENTION

### First Embodiment

A first embodiment of the invention will be described in detail by way of example with reference to the drawings. In the first embodiment, a basic posture evaluating apparatus according to embodiments of the invention is described.

(Configuration of Posture Evaluating Apparatus)

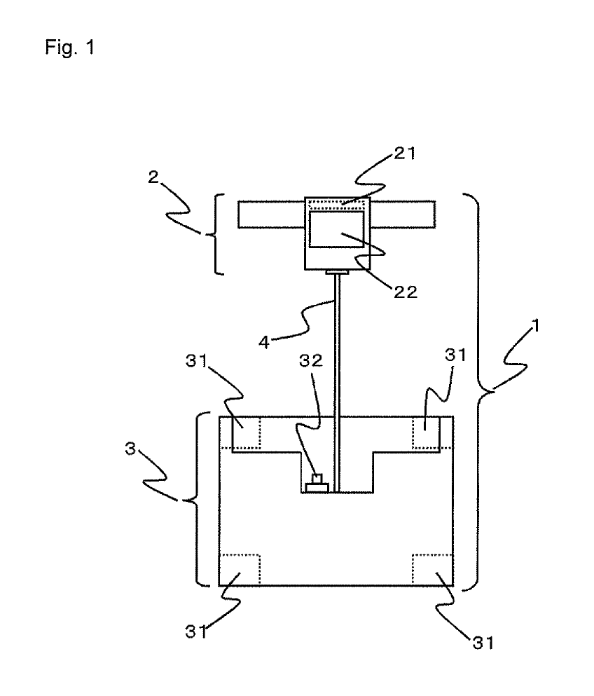

FIG. 1 is a schematic diagram of a posture evaluating apparatus 1 according to the first embodiment. The posture evaluating apparatus 1 includes a grasp portion 2 and a pedestal portion 3. The posture evaluation is measured in a state in which a user stands on the pedestal portion 3 (rides with both feet) while the grasp portion 2 is grasped with both hands. In the first embodiment, it is assumed that the posture evaluating apparatus is configured to be able to measure a weight of the user. Therefore, the pedestal portion 3 can also be called a weight scale unit.

The grasp portion 2 includes an inclination sensor 21 and a display panel 22. The pedestal portion 3 includes plural load sensors 31 and an accommodation state sensing unit 32.

The inclination sensor 21 is one that detects inclination of the grasp portion 2. An existing technique such as an inclination sensor in which an acceleration sensor is used may be applied to the inclination sensor 21. Any sensor that can detect the inclination should be included in the inclination sensor.

The display panel 22 is a display portion on which posture evaluation result and the like are displayed. Various display panels such as a liquid crystal display and an EL display can be applied to the display panel 22. In the first embodiment, the display panel 22 also acts as a touch panel type operation portion (operation panel), and the display panel 22 can perform operations such as on/off of a power supply, starting/interruption of the measurement, and input of physical information (such as body height, age, and sexuality). It is not always necessary that the touch panel be adopted in the operation portion, and the operation portion may be provided independently of the display panel. Part or the whole of the operation portion or display panel may be provided in the pedestal portion.

The load sensor 31 is one that detects a barycentric position of a load acting on the pedestal portion 3. An existing technique such as a strain gage may be applied to the load sensor 31. In the first embodiment, four load sensors 31 are provided in the pedestal portion 3.

The grasp portion 2 of the first embodiment is configured to be able to be accommodated in the pedestal portion 3. The accommodation state sensing unit 32 is a sensing portion that senses an accommodation state of the grasp portion 2. A contact switch that mechanically performs switching may be used as the accommodation state sensing unit 32, or a non-contact switch such as an optical sensor and a magnetic sensor may be used as the accommodation state sensing unit 32.

The grasp portion 2 and the pedestal portion 3 are configured such that a signal can be transmitted and received in a wireless or wired manner between the grasp portion 2 and the pedestal portion 3. In the example of FIG. 1, the grasp portion 2 and the pedestal portion 3 are connected through a cable 4, and a signal used in the measurement is transmitted and received through the cable 4 between the grasp portion 2 and the pedestal portion 3, and a driving power is supplied through the cable 4 between the grasp portion 2 and the pedestal portion 3.

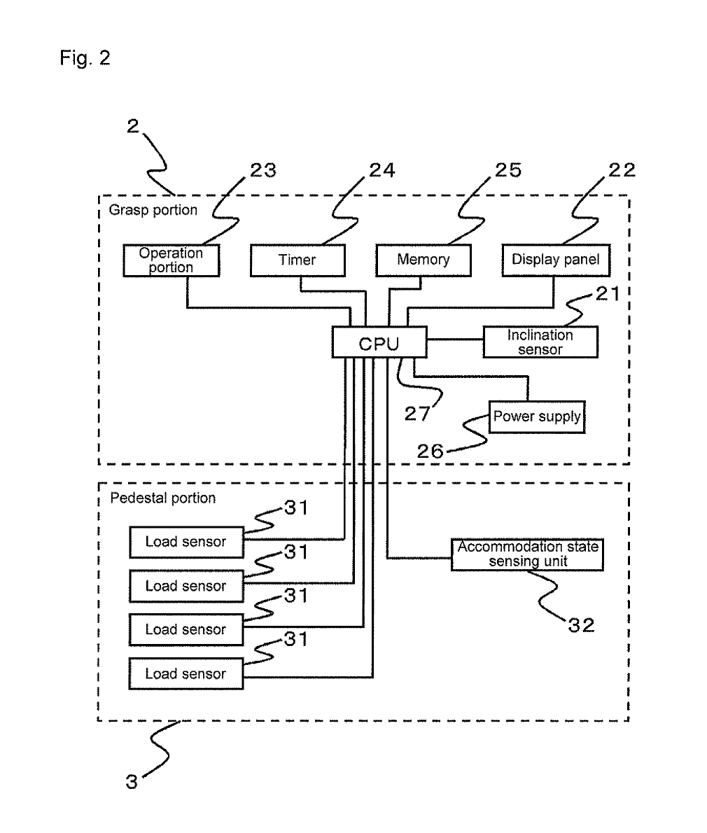

FIG. 2 is a block diagram illustrating an internal configuration of the posture evaluating apparatus 1. In FIG. 2, the component having the same function as that of FIG. 1 is designated by the same numeral, and the description is omitted. As illustrated in FIG. 2, the grasp portion 2 includes the inclination sensor 21, the display panel 22, an operation portion 23, a timer 24, a memory 25, a power supply 26, and a CPU (Central Processing Unit) 27. The pedestal portion 3 includes the load sensor 31 and the accommodation state sensing unit 32. The functions except the inclination sensor 21 may be provided in the pedestal portion 3.

The timer 24 is one that counts a measurement time and the like.

The memory 25 is one in which physical information, measurement (evaluation) result, and the like are stored. The memory 25 may be either a volatile memory or a nonvolatile memory.

The power supply 26 is one that supplies power to drive the grasp portion 2 and the pedestal portion 3. The power supply 26 may be either a household power supply or a battery and the like.

The CPU 27 is an operation processing portion that performs various pieces of operation processing based on the signal transmitted from each function of the posture evaluating apparatus 1. The CPU 27 specifically performs the following operations:
computing the inclination of the grasp portion 2 from the output signal of the inclination sensor 21,
computing the barycentric position of the load acting on the pedestal portion 3 from output signals of the plural load sensors 31,
computing the user weight from the output signals of the plural load sensors 31,
evaluating the user posture from the inclination of the grasp portion 2 and the barycentric position of the load acting on the pedestal portion 3, and
outputting results of the inclination of the grasp portion 2, the barycentric position of the load acting on the pedestal portion 3, the weight, the posture, and the like.

Although only one CPU 27 is illustrated in FIG. 2, plural CPUs may be provided. For example, the CPU may be provided in each kind of the operation.

(Measurement Flow)

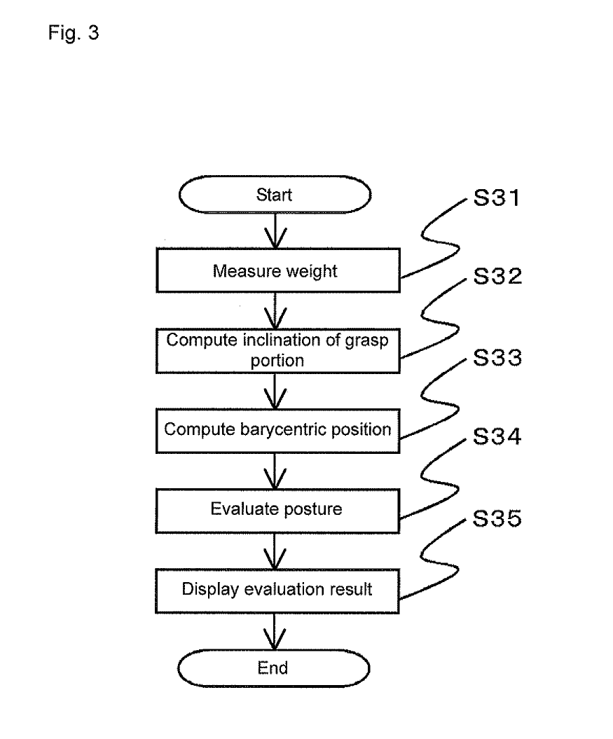

A measurement flow performed by the posture evaluating apparatus of the first embodiment will be described with reference to FIG. 3. FIG. 3 is a flowchart illustrating a measurement flow of the posture evaluating apparatus according to the first embodiment. It is assumed that the user stands on the pedestal portion 3 to grasp the grasp portion 2 with both hands. It is also assumed that the user intentionally keeps the grasp portion 2 in a horizontal position.

First the CPU 27 computes the user weight from the output signals of the plural load sensors 31 (Step S31). For example, the weight is a total of loads sensed by the load sensors 31. At this point, the computation result may be displayed on the display panel 22 in real time.

The CPU 27 computes the inclination of the grasp portion 2 from the output signal of the inclination sensor 21 (Step S32). Because the accurate evaluation result cannot be obtained when the user does not directly stick out both hands forward, the CPU 27 may previously display a message that “stick out directly both hands forward” or the like on the display panel 22. Therefore, it can be expected that the accurate evaluation result is obtained.

Then the CPU 27 computes the barycentric position of the load acting on the pedestal portion 3 from the output signals of the plural load sensors 31 (Step S33). For example, the barycentric position is computed from a magnitude relationship among the loads sensed by the load sensors 31. In the first embodiment, the barycentric position is measured while the user sticks out both hands forward. Desirably the user assumes a stiffly erect posture straight during the measurement of the barycentric position, because the barycentric position tends to be located rearward while the user sticks out both hands forward. Therefore, during the measurement of the barycentric position, the CPU 27 may display a message that the user should take both hands off from the grasp portion 2 to hold both hands to user's sides in an upright position on the display panel 22.

The CPU 27 evaluates the user posture from the inclination of the grasp portion computed in Step S32 and the barycentric position, which is computed in Step S33, of the load acting on the pedestal portion 3 (Step S34).

Then the CPU 27 displays the evaluation result in Step S34 on the display panel 22 (Step 535).

(Posture Evaluating Method and Evaluation Result Displaying Method)

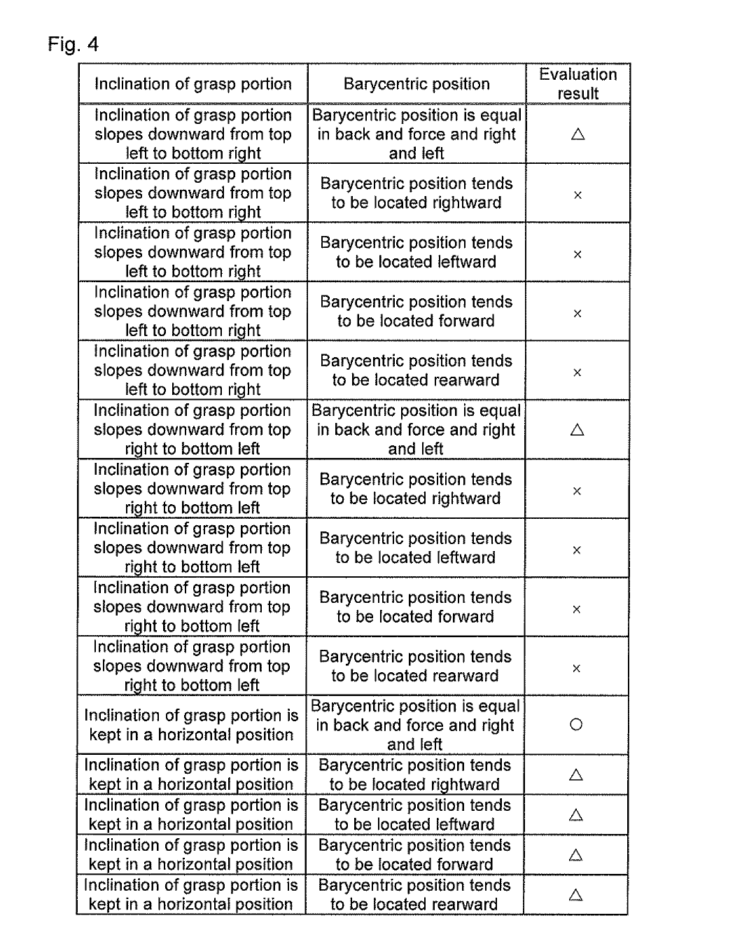

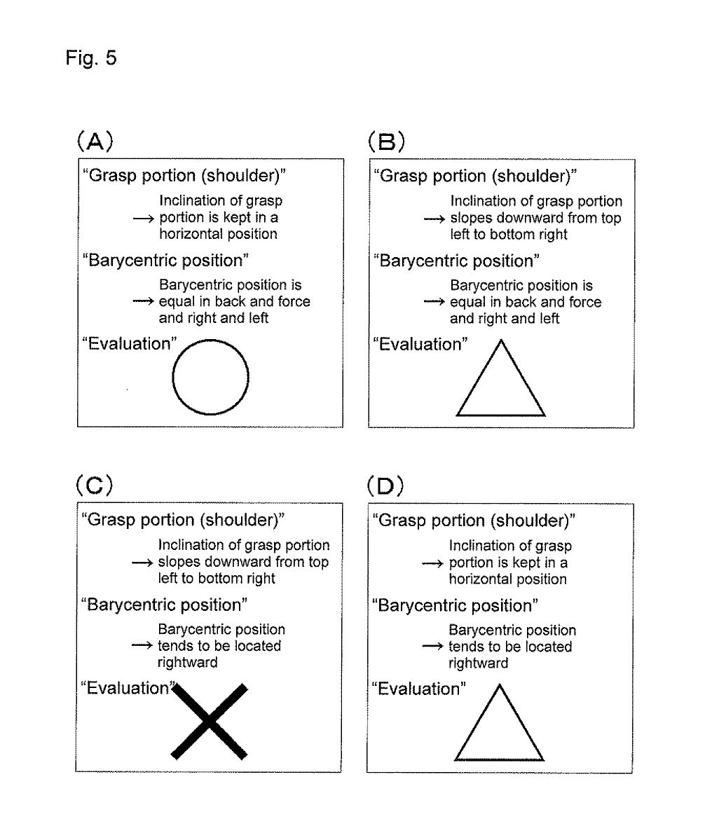

A posture evaluating method and an evaluation result displaying method will be described with reference to FIGS. 4 and 5. FIG. 4 is a view illustrating an example of a posture evaluating method performed by the posture evaluating apparatus according to the first embodiment. FIG. 5 is a view illustrating an example of an evaluation result displaying method performed by the posture evaluating apparatus according to the first embodiment.

In the example of FIG. 4, the posture evaluation is determined by a combination of the inclination of the grasp portion 2 and the barycentric position of the load acting on the pedestal portion 3. In the first embodiment, the posture is evaluated in three levels of “◯”, “Δ”, and “x” in the descending order of the posture. For example, the posture is evaluated in the level of “Δ” when “the inclination of grasp portion slopes downward from top left to bottom right while the barycentric position is equal in back and force and right and left”. Similarly, the posture is evaluated in the level of “x” when “the inclination of grasp portion slopes downward from top left to bottom right while the barycentric position tends to be located rightward”, the posture is evaluated in the level of “x” when “the inclination of grasp portion slopes downward from top left to bottom right while the barycentric position tends to be located leftward”, the posture is evaluated in the level of “x” when “the inclination of grasp portion slopes downward from top left to bottom right while the barycentric position tends to be located forward”, and the posture is evaluated in the level of “x” when “the inclination of grasp portion slopes downward from top left to bottom right while the barycentric position tends to be located rearward”. The posture is evaluated in the level of “Δ” when “the inclination of grasp portion slopes downward from top right to bottom left while the barycentric position is equal in back and force and right and left”, the posture is evaluated in the level of “x” when “the inclination of grasp portion slopes downward from top right to bottom left while the barycentric position tends to be located rightward”, the posture is evaluated in the level of “x” when “the inclination of grasp portion slopes downward from top right to bottom left while the barycentric position tends to be located leftward”, the posture is evaluated in the level of “x” when “the inclination of grasp portion slopes downward from top right to bottom left while the barycentric position tends to be located forward”, and the posture is evaluated in the level of “x” when “the inclination of grasp portion slopes downward from top right to bottom left while the barycentric position tends to be located rearward”. The posture is evaluated in the level of “◯” when “the inclination of grasp portion is kept in a horizontal position while the barycentric position is equal in back and force and right and left”, the posture is evaluated in the level of “Δ” when “the inclination of grasp portion is kept in a horizontal position while the barycentric position tends to be located rightward”, the posture is evaluated in the level of “Δ” when “the inclination of grasp portion is kept in a horizontal position while the barycentric position tends to be located leftward”, the posture is evaluated in the level of “Δ” when “the inclination of grasp portion is kept in a horizontal position while the barycentric position tends to be located forward”, and the posture is evaluated in the level of “Δ” when “the inclination of grasp portion is kept in a horizontal position while the barycentric position tends to be located rearward”.

Because the load acting on the pedestal portion is the user weight, the barycentric position of the load can be considered as the barycentric position of the user. In evaluating the posture based only on the barycentric position, the user posture is evaluated good (“◯”) when the barycentric position of the load acting on the pedestal portion is equal in back and force and right and left. However, even if the user's body strains (for example, right shoulder slopes downward), occasionally the barycentric position becomes the center depending on the way the user is standing. The posture evaluation performed only by the barycentric position makes a false evaluation (that the user posture is good).

Usually, for a person whose right shoulder slopes downward, the right hand goes down compared with the left hand even if the person sticks out both hands forward. Therefore, the inclination of the grasp portion slopes downward from top left to bottom right when the person grasps the grasp portion with both hands. That is, the inclination of the grasp portion can be considered as a deviation of the user's shoulder. In the first embodiment, the posture is evaluated in consideration of the inclination of the grasp portion, so that the posture is evaluated in the level of “Δ” when “the inclination of grasp portion slopes downward from top left to bottom right while the barycentric position is equal in back and force and right and left” (FIG. 4). Thus, the detailed evaluation result, which cannot be obtained only from the pedestal portion or the grasp portion, can be obtained in the posture evaluating apparatus of the first embodiment. Specifically, the false evaluation generated by the posture evaluation performed only by the pedestal portion can be reduced.

As illustrated in FIG. 5, the inclination of the grasp portion (shoulder), the barycentric position, and the posture evaluation (“◯”, “Δ”, and “x”) are displayed as the evaluation result on the display panel 22. The inclination of the grasp portion and the barycentric position may be displayed by numerical values or images expressing an inclination amount and a deviation amount.

As described above, in the first embodiment, the posture can be evaluated by the extremely simple method in which the user posture is evaluated based on the inclination of the grasp portion and the barycentric position of the load acting on the pedestal portion. The user posture is evaluated based on the inclination of the grasp portion and the barycentric position of the load acting on the pedestal portion, which allows the detailed evaluation result to be obtained compared with the posture evaluation that is performed only by the inclination of the grasp portion and the barycentric position of the load acting on the pedestal portion.

In the first embodiment, the posture evaluating apparatus includes not only the posture evaluation function but also the weight measuring function. Therefore, the user can recognize the own weight. Actually it is said that the body shape and the posture are related to each other, and it is believed that the body shape is improved by having the good posture. Therefore, the user can recognize the own weight to know effect of having the good posture. Additionally the user can recognize the own weight to have not only awareness of the posture improvement but also awareness of the body shape improvement.

### Second Embodiment

A second embodiment of the invention will be described in detail by way of example. In the second embodiment, a posture evaluating apparatus that evaluates the strain of the body (backbone) will be described as the posture evaluation. In the second embodiment, the description of the function similar to that of the first embodiment is omitted.

(Configuration of Posture Evaluating Apparatus)

Because a configuration of the posture evaluating apparatus of the second embodiment is similar to that (FIGS. 1 and 2) of the posture evaluating apparatus of the first embodiment, the description is omitted. However, the CPU 27 of the second embodiment further has a function of evaluating the strain of the body from the inclination of the grasp portion 2 and the barycentric position of the load acting on the pedestal portion 3.

(Measurement Flow)

Because a measurement flow of the posture evaluating apparatus of the second embodiment is similar to that (FIG. 3) of the posture evaluating apparatus of the first embodiment, the description is omitted.

(Posture Evaluating Method and Evaluation Result Displaying Method)

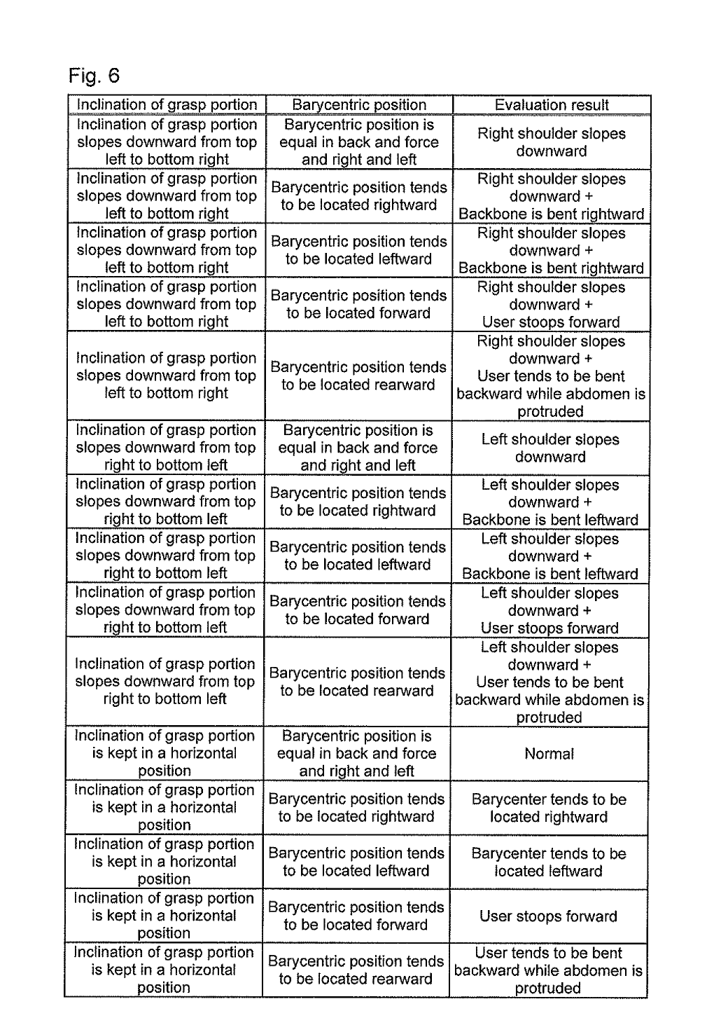

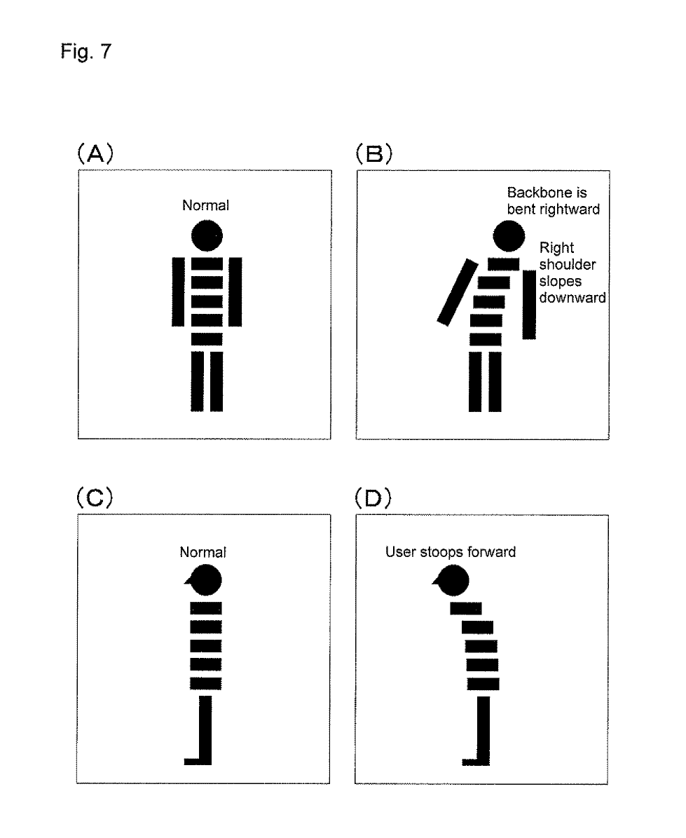

A posture evaluating method and an evaluation result displaying method will be described with reference to FIGS. 6 and 7. FIG. 6 is a view illustrating an example of a posture evaluating method performed by the posture evaluating apparatus according to the second embodiment. FIG. 7 is a view illustrating an example of an evaluation result displaying method performed by the posture evaluating apparatus according to the second embodiment.

As illustrated in FIG. 6, the posture evaluation performed by the posture evaluating apparatus of the second embodiment is determined by a combination of the inclination of the grasp portion 2 and the barycentric position of the load acting on the pedestal portion 3. In the example of FIG. 6, the posture that “the right shoulder slopes downward” is evaluated when “the inclination of the grasp portion from top left to bottom right while the barycentric position is equal in back and force and right and left”. Similarly, the posture that “the right shoulder slopes downward and the backbone is bent rightward” is evaluated when “the inclination of grasp portion slopes downward from top left to bottom right while the barycentric position tends to be located rightward”, the posture that “the right shoulder slopes downward and the backbone is bent rightward” is evaluated when “the inclination of grasp portion slopes downward from top left to bottom right while the barycentric position tends to be located leftward”, the posture that “the right shoulder slopes downward and the user stoops forward” is evaluated when “the inclination of grasp portion slopes downward from top left to bottom right while the barycentric position tends to be located forward”, and the posture that “the right shoulder slopes downward and the user tends to be bent backward while the abdomen is protruded” is evaluated when “the inclination of grasp portion slopes downward from top left to bottom right while the barycentric position tends to be located rearward”. The posture that “the left shoulder slopes downward” is evaluated when “the inclination of grasp portion slopes downward from top right to bottom left while the barycentric position is equal in back and force and right and left”, the posture that “the left shoulder slopes downward and the backbone is bent leftward” is evaluated when “the inclination of grasp portion slopes downward from top right to bottom left while the barycentric position tends to be located rightward”, the posture that “the left shoulder slopes downward and the backbone is bent leftward” is evaluated when “the inclination of grasp portion slopes downward from top right to bottom left while the barycentric position tends to be located leftward”, the posture that “the left shoulder slopes downward and the user stoops forward” is evaluated when “the inclination of grasp portion slopes downward from top right to bottom left while the barycentric position tends to be located forward”, and the posture that “the left shoulder slopes downward and the user tends to be bent backward while the abdomen is protruded” is evaluated when “the inclination of grasp portion slopes downward from top right to bottom left while the barycentric position tends to be located rearward”. That “the posture is normal” is evaluated when “the inclination of grasp portion is kept in a horizontal position while the barycentric position is equal in back and force and right and left”, the posture that “the barycenter is located rightward” is evaluated when “the inclination of grasp portion is kept in a horizontal position while the barycentric position tends to be located rightward”, the posture that “the barycenter tends to be located leftward” is evaluated when “the inclination of grasp portion is kept in a horizontal position while the barycentric position tends to be located leftward”, the posture that “the user stoops forward” is evaluated when “the inclination of grasp portion is kept in a horizontal position while the barycentric position tends to be located forward”, and “the user tends to be bent backward while the abdomen is protruded” is evaluated when “the inclination of grasp portion is kept in a horizontal position while the barycentric position tends to be located rearward”.

The evaluation result displayed on the display panel 22 may be the evaluation result expressed only by the characters or the evaluation result may be displayed by the image. As illustrated in FIG. 7, the evaluation result may be displayed by both the image and the characters. In the example of FIG. 7, the characters are displayed at a position corresponding to the image while the image corresponding to the evaluation result is displayed on the display panel 22. Specifically, the image of a person who stoops forward may be displayed when the evaluation result is “the user stoops forward”. The image of a person in which the right shoulder slopes downward while the backbone is bent rightward may be displayed when the evaluation result is “the right shoulder slopes downward and the backbone is bent rightward”, and the characters of “the right shoulder slopes downward” and the characters of “the backbone is bent rightward” may be displayed near the right shoulder of the image.

As described above, the strain of the body (backbone) is evaluated in the second embodiment. Therefore, the user can recognize the own posture in detail.

### Third Embodiment

A third embodiment of the invention will be described in detail by way of example. In the third embodiment, a posture evaluating apparatus that displays advice to have the good posture on the display panel according to the evaluation result. In the third embodiment, the description of the function similar to those of the first and second embodiments is omitted.

Because a configuration (FIGS. 1 and 2), a measurement flow (FIG. 3), and a posture evaluating method (FIGS. 4 and 6) of the posture evaluating apparatus of the third embodiment are similar to those of the posture evaluating apparatus of the first and second embodiments, the descriptions are omitted. However, the CPU 27 of the third embodiment displays the advice to have the good posture on the display panel 22 according to the evaluation result.

(Advice Displaying Method)

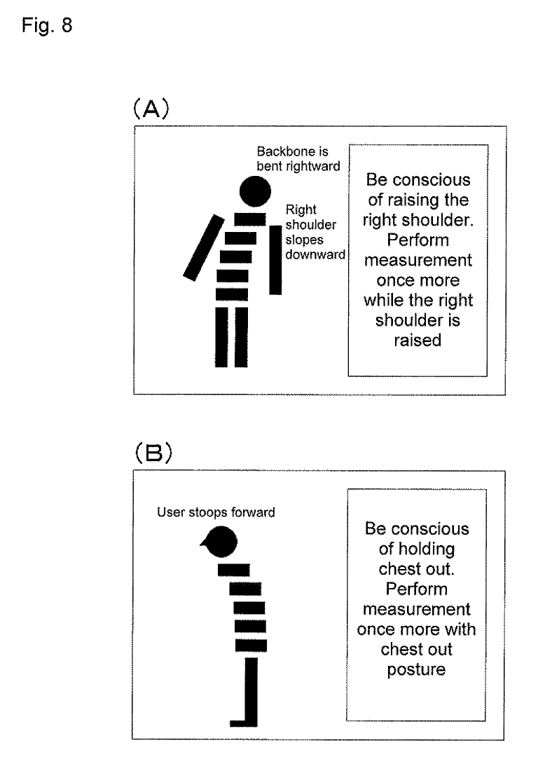

An advice displaying method will be described with reference to FIG. 8. FIG. 8 is a view illustrating an example of an advice displaying method performed by the posture evaluating apparatus according to the third embodiment. Although both the advice and the evaluation result are displayed on the display panel 22, it is not always necessary to display the evaluation result. Because an evaluation result displaying method of the third embodiment is similar to those of the embodiments, the description is omitted.

In the advice displaying method, for example, the evaluation result and the corresponding advice may previously be stored in the posture evaluating apparatus. The CPU 27 may select the advice corresponding to the evaluation result to display the selected advice on the display panel 22. For example, the advice may be a massage that “be conscious of raising the right shoulder” when the evaluation result is “the right shoulder slopes downward” or “the right shoulder slopes downward while the backbone is bent rightward”. Similarly, the advice may be a massage that “be conscious of holding chest out” when the evaluation result is “the user stoops forward”. The message that “the measurement may be performed once more while the right shoulder is raised” or the message that “the measurement may be performed once more with the chest out posture” may also be used as the advice. The advice triggers the re-measurement of the user. When performing the re-measurement, the user can recognize how much the conscious adjustment is required.

As described above, in the third embodiment, the advice to have the good posture is displayed on the display panel according to the evaluation result. Therefore, the user can recognize what the user should do in order to have the good posture.

### Fourth Embodiment

A fourth embodiment of the invention will be described in detail by way of example. In the fourth embodiment, a posture evaluating apparatus that includes not only the posture evaluation function but also the body composition estimating function will be described. In the fourth embodiment, the description of the function similar to those of the first to third embodiments is omitted.

(Configuration of Posture Evaluating Apparatus)

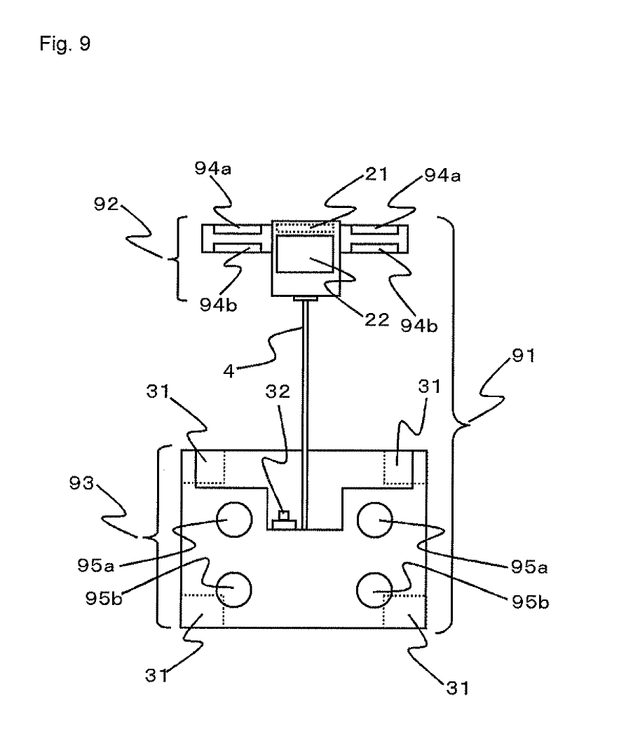

FIG. 9 is a schematic diagram of a posture evaluating apparatus 91 according to the fourth embodiment. In the posture evaluating apparatus 91, a grasp portion 92 includes hand electrodes 94a and 94b in addition to the functions possessed by the grasp portion 2 of FIG. 1. A pedestal portion 93 includes foot electrodes 95a and 95b in addition to the functions possessed by the pedestal portion 3 of FIG. 1.

The hand electrode 94a and the foot electrode 95a are electrodes used to pass currents through user's body. Hereinafter the hand electrode 94a and the foot electrode 95a are referred to as first electrodes. The hand electrode 94b and the foot electrode 95b are electrodes used to sense voltages (between hands or hand and foot). Hereinafter the hand electrode 94b and the foot electrode 95b are referred to as second electrodes.

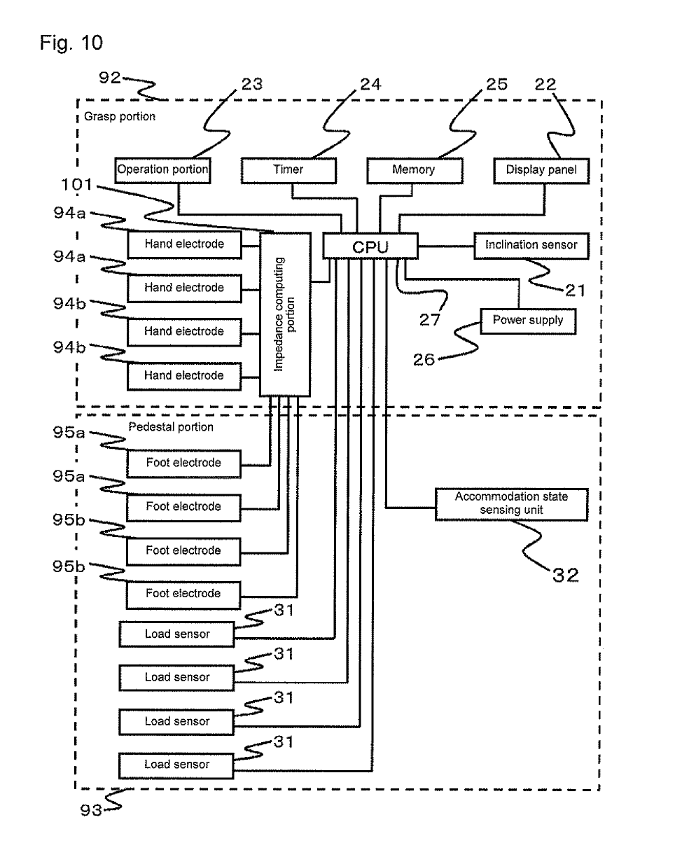

FIG. 10 is a block diagram illustrating an internal configuration of the posture evaluating apparatus 91. In FIG. 10, the same function as that of FIG. 9 is designated by the same numeral, and the description is omitted. As illustrated in FIG. 10, the grasp portion 2 further includes an impedance computing portion 101. The impedance computing portion 101 may be provided in the pedestal portion.

The impedance computing portion 101 is one that computes impedance. Specifically, the impedance computing portion 101 computes the impedance using current values passed from the first electrodes and the sensing result of the second electrodes.

The CPU 27 of the fourth embodiment also has a function of controlling the first electrodes and the second electrodes, a function of estimating the body composition from the computation result of the impedance computing portion 101, and a function of displaying the computation result on the display panel 22.

(Measurement Flow)

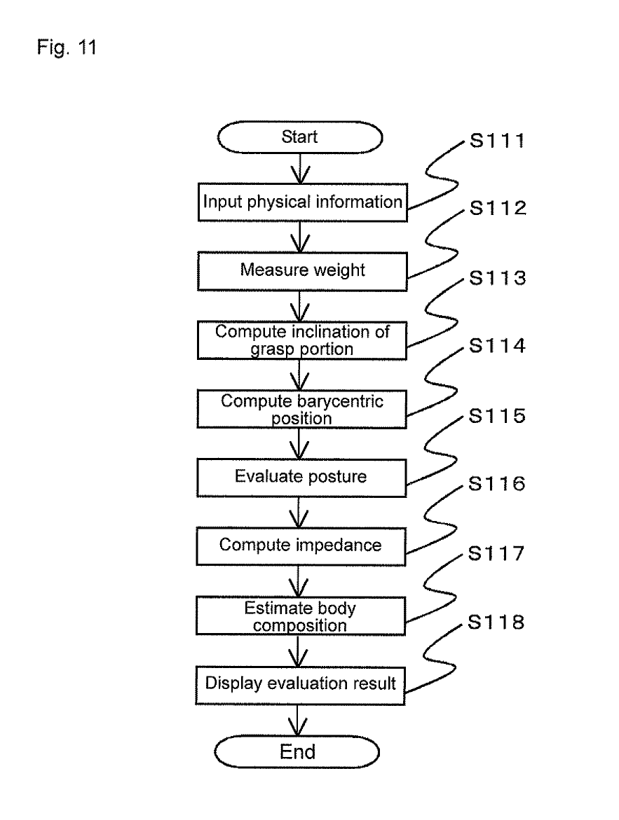

A measurement flow performed by the posture evaluating apparatus of the fourth embodiment will be described with reference to FIG. 11. FIG. 11 is a flowchart illustrating a measurement flow of the posture evaluating apparatus according to the fourth embodiment. It is assumed that the user stands on the pedestal portion 3 to grasp the grasp portion 2 with both hands. It is also assumed that the user intentionally keeps the grasp portion 2 in a horizontal position.

The CPU 27 performs display on the display panel 22 in order that the user is caused to input physical information (such as body height, age and sexuality) (Step S111). When the user inputs the physical information, the flow goes to Step S112.

Because processes in Steps S112 to S115 are similar to those in Steps S31 to S34 of FIG. 3, the descriptions are omitted.

(After Step S115) the CPU 27 passes the small current through the body from the first electrodes to sense the voltages with the second electrodes. The impedance computing portion 101 computes the impedance in user's body (Step S116).

The CPU 27 estimates the body composition of the user from the measurement result in Step S116 (Step S117).

Then the CPU 27 displays the evaluation result in Step S115 and the estimation result in Step S117 on the display panel 22 (Step S118).

As described above, in the fourth embodiment, the posture evaluating apparatus has not only the posture evaluating function but also the body composition estimating function. Therefore, the user can also obtain the information on the body composition such as the body fat percentage. Actually it is said that the body shape and the posture are related to each other, and it is believed that the body shape is improved by having the good posture. Therefore, the user can recognize the effect of having the good posture by obtaining the information on the body composition. Accordingly, the motivation in the improvement of the user posture can be expected to be enhanced. The user can be caused to have consciousness of not only the improvement of the posture but also the improvement of the body shape by obtaining the information on the body composition.

As described above, when the posture evaluating apparatus is formed by the configuration of the four embodiments, the user posture can easily be evaluated in detail, and the improvement of the user posture can effectively be assisted. The four embodiments can be combined as much as possible.

Although the four load sensors are used in the embodiments, any number of load sensors may be used.

The evaluation result of the posture is not limited to the results illustrated in FIGS. 4 and 6. For example, a backbone bending degree may be evaluated such that “the right shoulder slopes downward and the backbone is (slightly) bent rightward” when “the inclination of the grasp portion slopes downward from top left to bottom right while the barycentric position tends to be located rightward”, and such that “the right shoulder slopes downward and the backbone is (largely) bent rightward” when “the inclination of the grasp portion slopes downward from top left to bottom right while the barycentric position tends to be located leftward”.

Steps S31 to S33 of FIG. 3 may appropriately be changed. For example, the processes may be performed in the order of Step S33, Step S32, and Step S31. Steps S31 to S33 may simultaneously be performed when the CPU 27 can simultaneously perform the plural operations. Step S31 may be performed any time.

Steps S111 to S117 of FIG. 11 may appropriately be changed as long as Step S117 is performed after Step S111, Step S112, and Step S116.

The CPU 27 may display a degree of the inclination of the grasp portion 2 on the display panel 22 in real time. The CPU 27 may display the barycentric position of the load acting on the pedestal portion on the display panel 22 in real time. Therefore, the user can conducts training to have the good posture.

### DESCRIPTION OF SYMBOLS

1 posture evaluating apparatus
2 grasp portion
3 pedestal portion
4 cable
21 inclination sensor
22 display panel
23 operation portion
24 timer
25 memory
26 power supply
31 load sensor
32 accommodation state sensing unit
91 posture evaluating apparatus
92 grasp portion
93 pedestal portion
94a hand electrode
94b hand electrode
95a foot electrode
95b foot electrode
101 impedance computing portion

1. A posture evaluating apparatus comprising:
a grasp portion that is grasped with user's hands;
a pedestal portion on which the user rides with both feet;
inclination detecting means for detecting inclination of the grasp portion;
barycentric position detecting means for detecting a barycentric position of a load acting on the pedestal portion;
posture evaluating means for evaluating posture of the user based on the inclination of the grasp portion and the barycentric position of the load acting on the pedestal portion; and
output means for outputting evaluation result of the posture evaluating means.

2. The posture evaluating apparatus according to claim 1, wherein the barycentric position detecting means is a load sensor that is provided in the pedestal portion.

3. The posture evaluating apparatus according to claim 2, wherein the load sensor also acts as weight measuring means for measuring a weight of the user.

4. The posture evaluating apparatus as in claim 1, wherein the inclination detecting means is an inclination sensor that is provided in the grasp portion.

5. The posture evaluating apparatus as in claim 1, wherein the posture evaluating means evaluates strain of a body based on the inclination of the grasp portion and the barycentric position of the load acting on the pedestal portion.

6. The posture evaluating apparatus as in claim 1, wherein the output means outputs advice to have good posture according to the evaluation result of the posture evaluating means.

7. The posture evaluating apparatus as in claim 1, further comprising body composition estimating means for measuring impedance of the user to estimate a body composition from the impedance.

8. The posture evaluating apparatus as in claim 2, wherein the inclination detecting means is an inclination sensor that is provided in the grasp portion.

9. The posture evaluating apparatus as in claim 3, wherein the inclination detecting means is an inclination sensor that is provided in the grasp portion.

10. The posture evaluating apparatus as in claim 2, wherein the posture evaluating means evaluates strain of a body based on the inclination of the grasp portion and the barycentric position of the load acting on the pedestal portion.

11. The posture evaluating apparatus as in claim 3, wherein the posture evaluating means evaluates strain of a body based on the inclination of the grasp portion and the barycentric position of the load acting on the pedestal portion.

12. The posture evaluating apparatus as in claim 2, wherein the output means outputs advice to have good posture according to the evaluation result of the posture evaluating means.

13. The posture evaluating apparatus as in claim 3, wherein the output means outputs advice to have good posture according to the evaluation result of the posture evaluating means.

14. The posture evaluating apparatus as in claim 2, further comprising body composition estimating means for measuring impedance of the user to estimate a body composition from the impedance.

15. The posture evaluating apparatus as in claim 3, further comprising body composition estimating means for measuring impedance of the user to estimate a body composition from the impedance.
